(19) United States
(12) Patent Application Publication
(10) Pub. No.: US 2021/0386834 A1
(43) Pub. Date: Dec. 16, 2021

(54) COLLAGEN REPLACEMENT/REGENERATION FORMULATION AND USES THEREOF

(71) Applicant: Jose E Lopez Velarde Luna, Tepetlaoxtoc (MX)

(72) Inventor: Jose E Lopez Velarde Luna, Tepetlaoxtoc (MX)

(21) Appl. No.: 17/344,378

(22) Filed: Jun. 10, 2021

Related U.S. Application Data
(60) Provisional application No. 63/037,859, filed on Jun. 11, 2020.

(30) Foreign Application Priority Data
May 17, 2021 (MX) PCT/MX2021/050024

(51) Int. Cl. A61K 38/39; A61P 19/02; A61K 9/00; A61K 31/7008; A61K 31/202; A61K 31/375; A61K 31/737; A61K 31/728; A61K 33/32; A23L 33/125; A23L 33/17; A23L 33/16; A23L 33/12; A23L 33/00; A23K 20/163; A23K 20/147; A23K 20/20; A23K 20/158; A23K 50/40; A23K 50/20

(52) U.S. Cl. CPC A61K 38/39; A23V 2002/00; A61K 9/0056; A61K 31/7008; A61K 31/202; A61K 31/375; A61K 31/737; A61K 31/728; A61K 33/32; A23L 33/125; A23L 33/17; A23L 33/16; A23L 33/12; A23L 33/40; A23K 20/163; A23K 20/147; A23K 20/30; A23K 20/158; A23K 50/40; A23K 50/20; A61P 19/02

(57) ABSTRACT

Dietary supplements and formulations for a subject, including veterinary animals and humans, in an oral preparation and/or parenteral preparation, are provided. The formulations comprise elevated amounts of hydrolyzed collagen, a glucosamine salt, a glycosaminoglycan, a sulfone, a polysaccharide acid, an ascorbate salt, a transition metal, and a polyunsaturated fatty acid. A method for enhancing joint health and/or joint repair in a subject, including veterinary animals and humans, having a joint disorder or a disease associated therewith, is also provided, comprising administering the preparation comprising high amounts of hydrolyzed collagen.

### CROSS-REFERENCE TO RELATED APPLICATIONS

This application relates to foreign application No. PCT/MX2021/050024 filed 17 May 2021 and this application also claims the benefit of U.S. provisional application No. 63/037,859 filed 11 Jun. 2020, which is hereby incorporated by reference as though fully set forth herein.

### FIELD OF THE INVENTION

The present invention is related to the field of health nutraceutical and mineral supplement products, especially products and methods for promoting joint health, bone health, and/or anti-inflammation. The invention is also related to the field of veterinary nutraceuticals and methods.

### BACKGROUND OF THE INVENTION

Joint disorders and injuries are widespread, can cause considerable discomfort, and cost billions of dollars in lost days of work. Symptoms of these diseases and injuries include inflammation, lameness, loss of mobility, and pain.

Arthritis is a multifactorial degenerative joint disease, which progresses with age and results in joint stiffness, inflammation, and pain. There are two major types of arthritis, osteoarthritis, and rheumatoid arthritis.

Osteoarthritis is a condition occurring due to the progressive degeneration and the wearing away of the cartilage (the cushion between the joints), especially at the large joints like the hips and knees. The progressive degeneration of the joint due to osteoarthritis is irreversible. Present therapies are directed to palliative medical therapies to reduce inflammation and pain and surgical therapies to reconstruct an affected joint or, in severe cases, to replace the joint with an artificial, prosthetic joint.

Rheumatoid arthritis is a prostaglandin-mediated joint disease that leads to irreversible crippling of small joints, especially the fingers and toes. Essentially, the body's immune system attacks the cartilage, and the white blood cells (leukocytes) attack the collagen. Statin drugs, which are immunomodulators, are used frequently, though they have serious side effects. Temporary relief may be obtained from non-steroidal anti-inflammatory drugs (NSAIDs). However, overuse of these drugs can lead to ulcers.

Soft tissue rheumatism is a condition where many parts of the body can be affected for a variety of reasons. In many instances, soft tissue rheumatism is a sport-induced injury, such as a sprain, tennis elbow, or runner's knee. Soft tissues are the ligaments, tendons, and tendon sheath. Ligaments are bands of tissue that connect bones. Tendons are bands of tissue that connect muscle to bone. The tendon sheath is the tissue that surrounds and lubricates the tendon. Injury to any of these soft tissues can produce inflammation, pain, and stiffness. These conditions typically clear up quickly, within several days to weeks, and are usually treated with NSAIDs, icing the affected area, and rest.

In addition to the use of NSAIDs, the inflammatory response can be regulated through the use multiple other drugs. See, Goodman & Gilman's “The Pharmacological Basis of Therapeutics” eds. Hardman et al. Ninth Edition, McGraw-Hill Publishing, 1996. Unfortunately, certain anti-inflammatory drugs presently available produce cytotoxic effects that reflect their initial employment as cancer chemotherapeutics, typically anti-neoplastics. For example, corticosteroids, which are often used for treatment of acute inflammation, manifest significant adverse effects, such as inducing Cushingoid features, skin thinning, increased susceptibility to infection, and suppression of the hypothalamic-pituitary-adrenal axis.

Joint injuries and conditions as described above also afflict numerous other mammals, including domesticated animals such as dogs, cats, and horses. In particular, horses often sustain considerable joint injuries due to their participation in sporting events or use for farm work. Lameness due to traumatic joint disease is a common clinical problem in horses and is one of the most important sources of financial losses in the equine industry.

As described previously, such methods for treatment of both humans and animals only allow temporary relief and/or exhibit side effects from prolonged use. Therefore, there is a need for safe and effective treatment which can be used on a long-term basis without side effects and which also promotes rebuilding of the injured/diseased joints.

Hydrolyzed collagen is produced from hydrolysis of collagen derived from animal bones and skin. This process produces fragmented peptide protein derivatives that are more digestible, resulting in peptides and amino acids with a molecular weight of around 4000 Da which are more digestible for animals and may have an absorption of above 80%. Camacho-Zambrano, M. M., et al. (2009).

Glucosamine sulfate provides glucosamine molecules that are small and water soluble. These molecules improve joint functionality due to their inherent properties. Chondrocytes, or cartilage cells, also produce glucosamine from glucose as precursors of glycosaminoglycan units, which constitute one of the main components of articular cartilage, thus acting as a protector of cartilage. Previous studies have shown the effects of glucosamine sulfate on the degradation of animal cartilage. Osteoarthritis and Cartilage (2000) 8, 444-451, The effects of glucosamine derivatives on equine articular cartilage degradation in explant culture. J. I. Fenton, et al.

Chondroitin sulfate is a glycosaminoglycan most commonly found in the articular cartilage of adult horses. It is administered in the treatment of degenerative joint disease, and its effectiveness for treating animal joint damage is well documented. See, for example, Forsyth R K, Brigden C V, Northrop A J. Double blind investigation of the effects of oral supplementation of combined glucosamine hydrochloride and chondroitin sulphate on stride characteristics of veteran horses. Equine Vet J (Suppl) 36:622-5, 2006. Chondroitin sulfate has also been shown to inhibit degradative enzymes, including the metalloproteinases that destroy cartilage matrix. Bartolucci, C., “Chondroprotective action of chondroitin sulfate,” Int. J. Tiss. Reac., XIII(6):311-317 (1991). The sulfate group is essential to develop pharmacological and pharmacokinetic activity and also includes reparative benefits for joints and cartilage affected by osteoarthritis.

Hyaluronic acid exists as a naturally occurring polysaccharide (also known as a mucoid polysaccharide) that can be extracted from such diverse sources as rooster comb, umbilical cord, vitreous humor, synovial fluid, pathologic joints, skin and group A and C hemolytic Streptococci. Synovial fluid contains hyaluronic acid, and functions to lubricate joints to prevent friction between bones. Harsh impacts on animal joints may result in leakage of synovial fluid, so continuous stress to a joint can cause the progressive decrease and loss of this fluid.

Omega 3 fatty acids, when implemented in an equine diet, have shown effectiveness in helping to treat equine arthritis. See, for example, Manhart, D. & Scott, B. & Gibbs, P. & Coverdale, J. & Eller, E. & Honnas, C. & Hood, D. (2009). Markers of Inflammation in Arthritic Horses Fed Omega-3 Fatty Acids. The Professional Animal Scientist. 25. 10.15232/S1080-7446(15)30702-6.

Manganese acts as an activator of certain metabolic enzymes for horses, which require manganese as a part of their diet. See V. Juliand, W. Martin-Rosset. (2005). The growing horse: nutrition and prevention of growth disorders. 190. Magnesium is also a mineral that is involved in many enzymatic reactions in mammals. A. M. Johansson, et al. (2003). Hypomagnesemia in Hospitalized Horses. J. Vet. Intern. Med.; 17:860-867.

Vitamin C is a water-soluble antioxidant that plays a pivotal role in neutralizing harmful free radicals. Vitamin C also enhances immune system functionality by stimulating the formation of antibodies. Methylsulfonylmethane (MSM) is naturally occurring organosulfur compound, or a sulfone, that is important in the maintenance and conservation of connective tissue and in the correct lubrication of the joint, acting as a regenerator of articular cartilage.

There is yet to be developed an effective combination supplement for regenerating and healing articular cartilage, particularly those tissues in animals. The medical arts remain in need of such supplements, particularly in the veterinary arts for horses, canines, and other animals.

### SUMMARY OF THE INVENTION

The present invention meets the above and other important medical needs in the art. The present invention may be referred to as a dietary supplement or as a formulation.

An object of the present invention is to provide a formulation, dietary supplement and/or nutraceutical, comprising an oral preparation for treating mammals having joint disorders and/or diseases. The oral preparation may comprise a liquid, a powder, or a solid or semi-solid preparation, and may comprise a tablet, pill or capsule. In some embodiments, the oral preparation comprises a therapeutically effective amount of defined amounts of a combination of glucosamine salt, a glycosaminoglycan, a sulfone, hydrolyzed collagen, a polysaccharide acid, an ascorbate salt, a transition metal, and a polyunsaturated fatty acid.

Another object of the present invention is to provide a formulation, dietary supplement and/or nutraceutical which is a parenteral preparation for treating mammals having joint disorders or diseases, the parenteral preparation comprising a glucosamine salt, a glycosaminoglycan, a sulfone, hydrolyzed collagen, a polysaccharide acid, an ascorbate salt, a transition metal, and a polyunsaturated fatty acid.

Another object of the present invention is to provide a formulation or dietary supplement which is an intravenous preparation for treating mammals having joint disorders or diseases, the intravenous preparation comprising a glucosamine salt, a glycosaminoglycan, a sulfone, hydrolyzed collagen, a polysaccharide acid, an ascorbate salt, a transition metal, and a polyunsaturated fatty acid.

In still another object of the invention, a method for treating mammals having joint disorders and/or associated diseases by oral administration of a formulation comprising a glucosamine salt, a glycosaminoglycan, a sulfone, hydrolyzed collagen, a polysaccharide acid, an ascorbate salt, a transition metal, and a polyunsaturated fatty acid, is provided.

A further object of the present invention is to provide a method for treating mammals having joint disorders or diseases by parenteral administration of a formulation comprising a combination of a glucosamine salt, a glycosaminoglycan, a sulfone, hydrolyzed collagen, a polysaccharide acid, an ascorbate salt, a transition metal, and a polyunsaturated fatty acid.

A further object of the present invention is to provide a method for treating mammals having joint disorders or diseases by oral administration of a formulation comprising a combination of a glucosamine salt, a glycosaminoglycan, a sulfone, hydrolyzed collagen, a polysaccharide acid, an ascorbate salt, a transition metal, and a polyunsaturated fatty acid.

Yet another object of the present invention is to provide a method for treating mammals having joint disorders or diseases by intravenous administration of a formulation comprising a combination of a glucosamine salt, a glycosaminoglycan, a sulfone, hydrolyzed collagen, a polysaccharide acid, an ascorbic acid derivative, a transition metal, and a polyunsaturated fatty acid.

An additional object of the present invention is to provide a method for treating mammals having joint disorders or diseases by parenteral administration of a formulation comprising a glucosamine salt, a glycosaminoglycan, a sulfone, hydrolyzed collagen, a polysaccharide acid, an ascorbate salt, a transition metal, and a polyunsaturated fatty acid.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “derived from” shall be taken to indicate that a specified integer is obtained from a particular source, albeit not necessarily directly from that source.

A “composition” is intended to mean a combination of an active agent and another compound or composition, inert (for example, a detectable agent or label) or active, such as an adjuvant.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

As used herein, “about” is defined as including amounts varying from those stated by 5% to 10%.

Pharmaceutically acceptable salts include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, aspartic, benzoic, salicylic, oxalic, ascorbic acid, and the like) or bases (such as the hydroxide, carbonate or bicarbonate of a pharmaceutically-acceptable metal cation or organic cations derived from N,N-dibenzylethylenediamine, D-glucosamine, or ethylenediamine). The salts are prepared in a conventional manner, e.g., by neutralizing the free base form of the compound with an acid.

Other aspects and advantages of the present invention will become apparent from the subsequent detailed description and the appended claims. It is to be understood that one, some, or all of the properties of the various embodiments described herein may be combined to form other embodiments of the present invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

Oral Preparation: In some embodiments, the present formulation is an oral preparation comprising a glucosamine salt, a glycosaminoglycan, a sulfone, hydrolyzed collagen, a polysaccharide acid, an ascorbate salt, a transition metal, and a polyunsaturated fatty acid.

In one embodiment, the glucosamine salt in the present formulation may comprise glucosamine sulfate or glucosamine hydrochloride. In other embodiments, the present formulations and preparations comprise a single form of glucosamine, this being glucosamine sulfate.

In one embodiment, the glycosaminoglycan in the present formulation may comprise chondroitin sulfate.

In one embodiment, the sulfone in the present formulation may comprise methylsulfonylmethane (MSM).

In one embodiment, the polysaccharide acid in the present formulation may comprise hyaluronic acid. The hyaluronic acid may be derived from various sources, for example, animal or plant sources.

In one embodiment, the ascorbic acid derivative in the present formulation may comprise vitamin C and may be present as a pharmaceutically acceptable salt.

In one embodiment, the transition metal in the present formulation may comprise manganese. In another embodiment, the transition metal in the present formulation may comprise magnesium.

In one embodiment, the polyunsaturated fatty acid in the present formulation may comprise an omega-3 fatty acid, including, by way of example, alpha-linolenic acid, eicosapentaenoic acid, or docosahexaenoic acid. In other embodiments, the polyunsaturated fatty acid in the present formulation may comprise an omega-6 fatty acid, or an omega-9 fatty acid.

In some embodiments, the oral formulation is a powder which may be mixed with food for administration to an animal.

The present oral formulations as described herein may be prescribed without adding flavorants or the like. Such is provided because, among other reasons, the formulations are palatable to an animal without additional flavorants, eliminating the need to combine with animal feed, forage or other agents for administration. These formulations do not possess a bitter taste and are not otherwise unpalatable to an animal, providing an additional advantage.

In another embodiment, the formulation may be prepared with a flavorant and/or an excipient.

A variety of commonly used excipients in dietary supplement formulations may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste-masking agents, an effervescent disintegration agent, and combinations of any of these agents.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacryl amides, polyvinyloxoazolidone, polyvinylalcohols, C12-C18 fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another embodiment, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid.

Parenteral Preparation: In some embodiments, the present formulation is a parenteral preparation. By way of example, the parenteral preparation may comprise a combination of a glucosamine salt, a glycosaminoglycan, a sulfone, hydrolyzed collagen, a polysaccharide acid, an ascorbate salt, a transition metal, and a polyunsaturated fatty acid.

Method of Treatment: In some aspects, a method is providing for regenerating and/or improving collagen at a joint in a subject is provided. In some embodiments, the method comprises administering a formulation comprising a combination of a glucosamine salt, a glycosaminoglycan, a sulfone, hydrolyzed collagen, a polysaccharide acid, an ascorbate salt, a transition metal, and a polyunsaturated fatty acid, to the subject. In some embodiments, the composition is administered by combining the composition into a foodstuff of the subject, such as in the feed of an animal. By way of example, the animal may be a horse, dog, cow, pig, or other animal.

In another embodiment, the method may comprise the formulation being administered via intramuscular injection. In another embodiment, the method may comprise the formulation being administered in the skin by subcutaneous injection. In still another embodiment, the method may comprise the formulation being administered via epidural injection. In yet another embodiment, the method may comprise the formulation being administered via intrathecal injection. In another embodiment, the method may comprise the formulation being administered via intraosseous injection.

In another embodiment, the method may comprise administering the formulation via an intravenous administration of a formulation. The formulation will be prepared so as to be physiologically acceptable for IV administration, and to include those ingredients that render the formulation suitable for IV administration to the animal, and will also include a glucosamine salt, a glycosaminoglycan, a sulfone, hydrolyzed collagen, a sulfone, a polysaccharide acid, an ascorbate salt, a transition metal, and a polyunsaturated fatty acid.

### DESCRIPTION OF THE SEVERAL VIEWS OF THE DRAWINGS

FIG. 1 illustrates a table showing a comparison of one embodiment of the oral formulation with other products that are currently marketed for animals such as horses. FIG. 1 includes the amounts of certain key ingredients for each product, as well as a listing of additional ingredients that are not found in the embodied oral formulation. As shown in FIG. 1, none of the marketed products have all of the compounds found in the embodiment of the oral formulation.

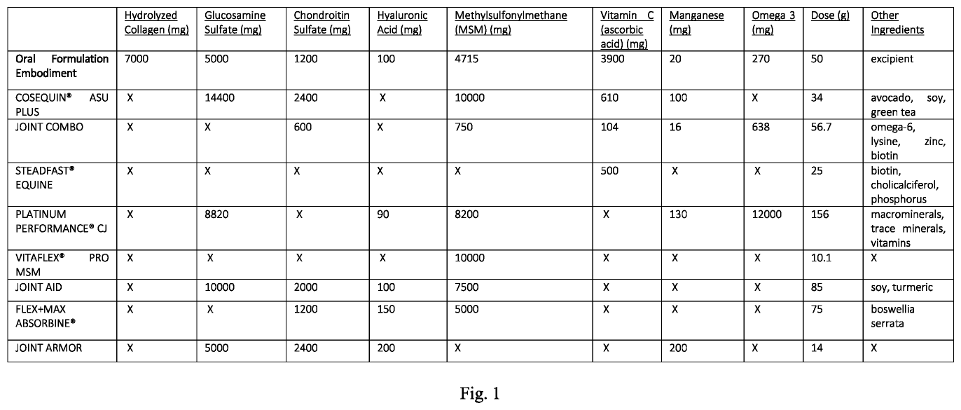

FIG. 2 illustrates a table showing a comparison of the ingredient weight fraction in one embodiment of the oral formulation with other products that are currently marketed for animals such as horses. The weight fraction is calculated by dividing the ingredient amount for the respective formulation by its respective recommended dosage.

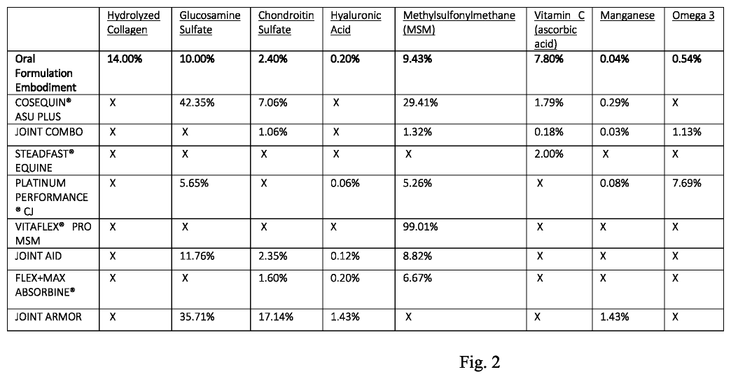

FIG. 3 illustrates a table showing a comparison of an alternative embodiment of the oral formulation with other products that are currently marketed for animals such as horses. As shown in FIG. 3, none of the marketed products have all of the compounds found in the alternative embodiment of the oral formulation.

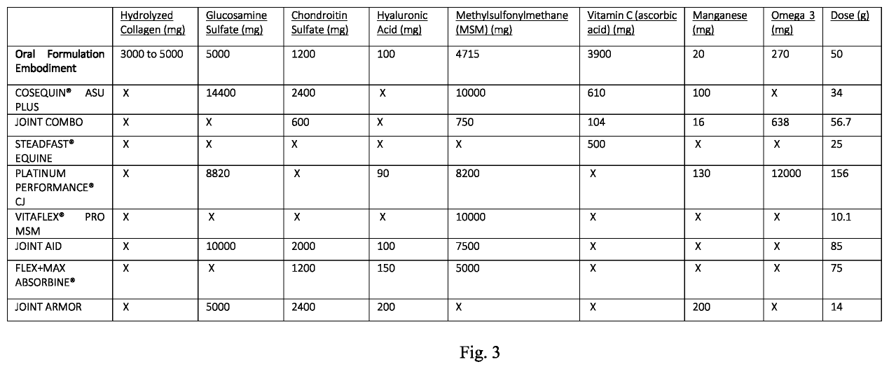

### DETAILED DESCRIPTION OF THE INVENTION

The following examples present a description of various specific aspects of the intended invention, and are not presented to limit the intended invention in any way.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

### Formulations

Various embodiments of the present oral formulation include a combination of ingredients that may be defined by weight fraction. Examples of these weight fraction percentages in the formulation are presented below.

Oral Formulation: In embodiments, the oral formulation ingredients may comprise hydrolyzed collagen, glucosamine sulfate, chondroitin sulfate, hyaluronic acid, MSM, vitamin C, manganese, and an omega-3 fatty acid. The amount of each ingredient may be based on a weight fraction calculation wherein the oral formulation has a 50 gram weight dosage.

In embodiments, the weight fraction of glucosamine sulfate may be about 25% or less, about 20% or less, about 15% or less, or about 10% or less of the total weight of the supplement composition.

The weight fraction of hydrolyzed collagen may be about 35% or less, about 30% or less, about 25% or less, about 20% or less, about 15% or less, about 10% or less, or about 5% or less of the total weight of the supplement composition. The present formulation is not essentially free of hydrolyzed collagen.

The weight fraction of MSM may be about 25% or less, about 20% or less, about 15% or less, or about 10% or less of the total weight of the supplement composition. The present formulation is not essentially free of MSM.

The weight fraction of manganese may be about 0.5% or less, about 0.1% or less, or about 0.05% or less of the total weight of the supplement composition.

In other embodiments, magnesium is used in conjunction with or in place of manganese. The weight fraction of magnesium may be about 0.5% or less, about 0.1% or less, or about 0.05% or less of the total weight of the supplement composition.

The weight fraction of chondroitin sulfate may be about 10% or less, about 6% or less, about 4% or less, or about 2% or less of the total weight of the supplement composition. The present formulation is not essentially free of chondroitin sulfate.

The weight fraction of hyaluronic acid may be about 1.0% or less, about 0.5% or less, about 0.2%, or about 0.1% or less of the total weight of the supplement composition. The present formulation is not essentially free of hyaluronic acid.

The weight fraction of vitamin C may be about 20% or less, about 15% or less, about 10% or less, or about 5% or less of the total weight of the supplement composition. The present formulation is not essentially free of vitamin C.

The weight fraction of the omega-3 fatty acid may be about 2.0% or less, about 1.0% or less, or about 0.5% or less of the total weight of the supplement composition. The present formulation is not essentially free of an omega-3 fatty acid.

In one embodiment, the oral formulation may be described as comprising a combination of ingredients based on the individual ingredient weight to be included in a 50 gram dose of the composition prepared as an oral preparation. For example, a 50 g dose of the oral formulation may comprise a combination of about 5000 mg glucosamine sulfate; about 3900 mg ascorbic acid; about 270 mg of omega-3 fatty acid; about 4715 mg MSM; about 1200 mg chondroitin sulfate; about 20 mg manganese; about 7000 mg hydrolyzed collagen; and about 100 mg hyaluronic acid, wherein the balance of the formulation may comprise one or more excipients.

Parenteral Formulation: The parenteral formulation in some embodiments may comprise a combination of the above ingredients wherein the glycosaminoglycan is hyaluronan, chondroitin, dermatin, keratin, heparan, or heparin, or pharmaceutically acceptable salts of these compounds.

In some embodiments, the formulations and compositions include a glycosaminoglycan in an amount of about 0.01 mg/kg to about 0.5 mg/kg. In some embodiments, when the glycosaminoglycan or a pharmaceutically acceptable salt thereof is administered intravenously, the glycosaminoglycan or a pharmaceutically acceptable salt thereof can be administered at a dose from about 0.01 to about 0.1 mg/kg. In some embodiments, the glycosaminoglycan is dissolved in hyaluronic acid in an amount from about 0.001 mg/kg to about 0.1 mg/kg. In certain embodiments, the parenteral formulation may comprise a mixture including between 0.05 to 1% hyaluronic acid, 1% to 10% of a glycosaminoglycan, and 0.05 to 1% of hydrolyzed collagen. In one embodiment, the glycosaminoglycan is chondroitin sulfate. In one embodiment, the subject is being treated for osteoarthritis. In one embodiment, the subject is being treated for a lameness or other loss of articular functionality.

In yet another embodiment, the parenteral formulation may be administered intravenously. In another embodiment, the parenteral formulation may be administered intramuscularly.

In one embodiment, the parenteral formulation may be in the form of a sterile aqueous solution which may contain other substances, for example enough salts or monosaccharides to make the solution isotonic with blood.

The term “carrier” refers to compositions of matter that are conventionally used in the art to facilitate the storage, administration, and/or the biological activity of an active compound. See, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the parenteral formulation may include those conventionally used, e.g., water, saline, aqueous dextrose, lactose, a buffered solution, hyaluronan and glycols. Suitable pharmaceutical carriers include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

### EXAMPLES

The present invention is not to be limited in scope by the specific examples described herein. Functionally equivalent products, compositions and methods are clearly within the scope of the invention, as described herein.

### Example 1—Cartilage Regeneration Via Oral Administration

The following is one example demonstrating the utility of the present invention for regenerating cartilage using one embodiment of the present formulation.

The present example demonstrates the regeneration of cartilage in an animal, particularly a horse, and more particularly a knee joint.

In this and other examples, the degree of lameness and pain may be evaluated using the Modified Obel Lameness Score. See Orsini J, Divers T. Management of special problems. Manual of Equine Emergencies, Treatment and Procedures. Philadelphia, Pa.: Saunders; 2003:749, 750. At grade 0 on the scale, a horse shows no gait abnormalities at a walk or trot. At grade 1 on the scale, the horse shows foot lifting at rest, a normal gait when walking, but a shortened stride, even head and neck lifting for each foot while trotting. At grade 2 on the scale, the walk was stilted, but there was no abnormal head or neck lifting; the trot showed obvious lameness with uneven head and neck lifting. At grade 3, lameness is obvious during a walk or trot, and the horse may resist attempts to have a forefoot lifted or show a reluctance to move. At grade 4, the horse has difficult bearing weight at rest or is reluctant to move.

In other examples, lameness may be evaluated using the scale provided by the American Association of Equine Practitioners (AAEP). See Anon (1991) Lameness scale. Definition and classification of lameness. American Association of Equine Practitioners. 19. At grade 0 on the scale, no lameness is perceptible under any circumstances. At grade 1 on the scale, lameness is difficult to observe and is not consistently apparent. At grade 2 on the scale, lameness is difficult to observe at a walk or when trotting in a straight line but consistently apparent under certain circumstances (e.g. weight-carrying, circling, inclines, hard surface). At grade 3, lameness is consistently observable at a trot under all circumstances. At grade 4, lameness is obvious at a walk. At grade 5, lameness produces minimal weight bearing in motion and/or at rest or a complete inability to move.

Prior to treatment, the horse underwent a routine lameness examination, including a flexion test for the front and hind legs. Flexion testing demonstrated pain for the horse's front right leg at the knee and ankle joints. The lameness examination showed a noticeable limp by the horse and a clear favoring of its left side due to joint damage on the right side, which indicated a Modified Obel Lameness grade of at least 3.

The horse was administered an oral formulation comprising about 14% hydrolyzed collagen, 10% glucosamine sulfate, 2.4% chondroitin sulfate, 0.2% hyaluronic acid, 9.4% MSM, 7.8% ascorbic acid, 0.04% manganese, and 0.5% omega-3 fatty acid. The oral formulation was administered for four months, and the horse ingested 50 g of the oral formulation once per day during the testing period.

Following one month of oral intake of the formulation, the horse showed decreased pain during lameness and flexion testing. While jogging, the horse demonstrated a decreased favoring of the left side and soundness evidencing less limping compared with the time period prior to administration of the oral formulation. The Modified Obel Lameness observably decreased to grade 1.

Following four months of treatment, x-rays showed cartilage regeneration in the affected joint. Measurements showed an approximately 5.0 mm layer of cartilage in the knee joint after four months. Prior to treatment, cartilage was present in trace amounts in the knee, but no greater than 0.5 mm in thickness.

### Example 2—Alleviation of Arthritis Symptoms Via Oral Administration

The present example demonstrates alleviation of arthritis symptoms in the joints of an animal, more particularly a horse.

In this example the subject is a horse. Prior to treatment, the horse in this example underwent a hoof tester examination, which indicated that the horse had deep navicular pain in its right front leg. The horse also showed a favoring of its left side due to joint damage on the right side, and a lameness examination indicated a Modified Obel Lameness grade of at least 3 while the horse trotted or walked.

The horse was given an oral formulation comprising about 14% hydrolyzed collagen, 10% glucosamine sulfate, 2.4% chondroitin sulfate, 0.2% hyaluronic acid, 9.4% MSM, 7.8% ascorbic acid, 0.04% manganese, and 0.5% omega-3 fatty acid. The oral formulation was administered for four weeks, and the horse ingested 50 g of the oral formulation once per day during the testing period.

Following four weeks of intake of the oral formulation, the horse showed no evidence of navicular pain during a hoof tester examination. While jogging, the horse demonstrated a decreased favoring of the left side and soundness evidencing less limping compared with the period prior to administration of the oral formulation. The Modified Obel Lameness observably decreased to grade 1.

### Example 3—Cartilage Regeneration Via Oral Administration

The following is one example demonstrating the utility of the present invention for regenerating cartilage using one embodiment of the present formulation.

The present example demonstrates the regeneration of cartilage in an animal, particularly a horse, and more particularly a knee joint.

This horse was 17 years old prior to beginning treatment with the oral formulation. This horse had also competed in events at the Pan American Games and was regularly involved in highly strenuous and athletic activity.

Prior to treatment, the horse underwent a routine lameness examination that showed a noticeable limp by the horse and a clear favoring of its left side due to joint damage on the right side.

The horse was administered an oral formulation comprising about 14% hydrolyzed collagen, 10% glucosamine sulfate, 2.4% chondroitin sulfate, 0.2% hyaluronic acid, 9.4% MSM, 7.8% ascorbic acid, 0.04% manganese, and 0.5% omega-3 fatty acid. The oral formulation was administered for three years, and the horse ingested 50 g of the oral formulation once per day during the testing period.

After three years of continuous treatment of the oral formulation, x-rays showed cartilage regeneration in the affected joints for the horse's front leg. Measurements showed an approximately 3.0 mm layer of cartilage in the front leg joints. Prior to treatment, cartilage was present in trace amounts in the joints, but no greater than 0.5 mm in each joint.

### Example 4—Improved Soundness for Horse Following Oral Administration

The following is one example demonstrating the utility of the present invention for improving the soundness in an animal, particularly a horse.

The horse in this example was 15 years old prior to beginning treatment with the oral formulation. This horse had a medical history that included a medical diagnosis at 8 years old of degenerative joint disease in the left and right coffin joints, enlarged dorsal spurs, and bone spurs in its navicular proximal flexor articulations.

Prior to treatment, the horse underwent a routine lameness examination that showed a low level of soundness based on the horse's flight path and trotting ability due to the joint disease. A lameness examination indicated an AAEP Lameness grade of at least 3 while the horse trotted or walked, evidencing obvious lameness.

The horse was administered an oral formulation comprising about 14% hydrolyzed collagen, 10% glucosamine sulfate, 2.4% chondroitin sulfate, 0.2% hyaluronic acid, 9.4% MSM, 7.8% ascorbic acid, 0.04%, and 0.5% omega-3 fatty acid. The oral formulation was administered for 31 days, and the horse ingested 50 g of the oral formulation once per day during the testing period.

A soundness examination was conducted following the 31-day period of administering the oral formulation. The examination demonstrated improved soundness and briskness of pace, and a lameness examination showed that the AAEP Lameness grade decreased to 1, indicating an improvement in soundness. Observation of the left hind portions, including the gluteal muscles, the biceps femoris, and the semitendinous, showed an increased size for these muscles. The biceps femoris increased in circumference following the administration of the oral formulation.

### Example 5—Improved Soundness for Canine Following Oral Administration

The following is one example demonstrating the utility of the present invention for improving the soundness in an animal, particularly a canine.

The canine was an 11 year-old Yorkie with a medical history that included a cruciate ligament tear and advanced arthritis in its left hind leg.

Prior to treatment, the canine underwent a routine lameness examination that showed a low level of soundness and complete inability to place any weight on the left hind leg.

The canine was administered an oral formulation comprising about 14% hydrolyzed collagen, 10% glucosamine sulfate, 2.4% chondroitin sulfate, 0.2% hyaluronic acid, 9.4% MSM, 7.8% ascorbic acid, 0.04% manganese, and 0.5% omega-3 fatty acid. The oral formulation was administered for 30 days, and the canine ingested 50 g of the oral formulation once per day during the testing period. The canine also underwent non-invasive laser therapy treatment for its nervous system as part of the recovery process.

A conventional tool used in assessing reduced pain is to assess the speed at which an animal is able to trot over a given period of 30 seconds. (The Trot Test). For example, this tool has been used to assess soundness and joint diseases in canines. For a canine, the “trotting” test may take the form of assessing the distance a canine can trot in a 1 minute period, after having been on a treatment regimen of the present formulation as a feed additive (at a dose of 0.6 g/kg, for example) for a period of time of at least 1 month. An improvement will be identified in an animal that shows an improvement as measured by the “Trot Test” of at least 20%.”

A Trotting Test was conducted on the canine following a 12-day period of administering the oral formulation. The examination demonstrated improved soundness, briskness of pace, and recovery of use for the left hind leg. Following a 30-day period of ingesting the oral formulation, the canine had completely regained use and functionality of its left hind leg and drastically improved its soundness compared to the time period prior to treatment. The canine's trotting pace increased by twice the speed that the canine could maintain prior to administration of the oral formulation.

### Example 6—Collagen Regeneration Via Intravenous Administration

The present example demonstrates the utility of the present invention for regenerating collagen using one embodiment of the present formulation.

A formulation of the present preparations may be prepared that is suitable for intravenous administration employing techniques well known to those of ordinary skill in the medical arts. The preparations will include, among other things, an amount of hydrolyzed collagen sufficient to deliver a dosage of this ingredient that is essentially equivalent to the amount of hydrolyzed collagen provided to an animal administered a 50 gram oral dose of the formulation comprising about 7,000 mg of hydrolyzed collagen. An intravenous-administered preparation typically provides a greater percentage of the active ingredient to the animal, compared to an oral administration. Therefore, the intravenous formulation of the present invention in some embodiments will include a lower concentration of hydrolyzed collagen compared to amount of hydrolyzed collagen provided in the oral preparation.

### Example 7—Powder Oral Formulation

The following illustrates one example of the present invention and a table comparing ingredients in the same.

TABLE 1
Comparison of Oral Formulation Embodiment With Other Products

| | | | | | | Vitamin | | | | |
|---|---|---|---|---|---|---|---|---|---|---|
| | | | | | | C | | | | |
| | Hydrolyzed | Glucosamine | Chondroitin | Hyaluronic | Methyl- | (ascorbic | | Omega | | |
| | Collagen | Sulfate | Sulfate | Acid | sulfonylmethane | acid) | Manganese | 3 | Dose | Other |
| | (mg) | (mg) | (mg) | (mg) | (MSM) (mg) | (mg) | (mg) | (mg) | (g) | Ingredients |
| Oral | 7000 | 5000 | 1200 | 100 | 4715 | 3900 | 20 | 270 | 50 | excipient |
| Formulation | | | | | | | | | | |
| Embodiment | | | | | | | | | | |
| COSEQUIN ® | X | 14400 | 2400 | X | 10000 | 610 | 100 | X | 34 | avocado, |
| ASU PLUS | | | | | | | | | | soy, green |
| | | | | | | | | | | tea |
| JOINT | X | X | 600 | X | 750 | 104 | 16 | 638 | 56.7 | omega-6, |
| COMBO | | | | | | | | | | lysine, zinc, |
| | | | | | | | | | | biotin |
| STEADFAST ® | X | X | X | X | X | 500 | X | X | 25 | biotin, |
| EQUINE | | | | | | | | | | cholecalciferol, |
| | | | | | | | | | | phosphorus |
| PLATINUM | X | 8820 | X | 90 | 8200 | X | 130 | 12000 | 156 | macrominerals, |
| PERFORMANCE ® | | | | | | | | | | trace |
| CJ | | | | | | | | | | minerals, |
| | | | | | | | | | | vitamins |
| VITAFLEX ® | X | X | X | X | 10000 | X | X | X | 10.1 | |
| PRO MSM | | | | | | | | | | |
| JOINT AID | X | 10000 | 2000 | 100 | 7500 | X | X | X | 85 | soy, |
| | | | | | | | | | | turmeric |
| FLEX + MAX | X | X | 1200 | 150 | 5000 | X | X | X | 75 | boswellia |
| ABSORBINE ® | | | | | | | | | | serrata |
| JOINT | X | 5000 | 2400 | 200 | X | X | 200 | X | 14 | |
| ARMOR | | | | | | | | | | |

The present formulation having the above components was prepared in a powder formulation.

The dose amount of the present formulation may vary depending upon the informed opinion of an attending medical professional. The dosage may be based on the size and/or type of animal being treated, or the disorder being treated.

The amount of any ingredient in the oral formulation dose may also be changed based on the size and/or type of animal being treated, or the disorder being treated.

In some embodiments, the present formulation does not include ingredients that are considered “other ingredients” that are present in other formulations, and are essentially free of these ingredients. Examples of “other ingredients” include lysine, zinc, green tea, soy, flavorings, turmeric, biotin, or Boswellia serrata, among others. This poses an advantage for the present formulation of avoiding possibly adverse side effects in a subject from the ingredient, such as allergies, as well as any pre-existing health condition. In other embodiments, the formula is created to avoid any allergenic reaction risk to the animal.

In some embodiments, the oral formulation is a dry powder that may comprise, per 50 g of the dry powder, a combination of about 5000 mg glucosamine sulfate; about 3900 mg ascorbic acid; about 270 mg of omega-3 fatty acid; about 4715 mg MSM; about 1200 mg chondroitin sulfate; about 20 mg manganese; about 7000 mg hydrolyzed collagen; and about 100 mg hyaluronic acid.

As demonstrated in Table 1 above, the present forms are not essentially free of hydrolyzed collaged, are not essentially free of chondroitin sulfate, are not essentially free of glucosamine sulfate, are not essentially free of hyaluronic acid, and/or are not essentially free of MSM.

The following Table 2 presents a comparison of the following products by weight percentage. These percentages were calculated by using the ingredient amounts and doses provided in Table 1.

TABLE 2
Comparison of Oral Powder Formulation Embodiment With Other Products by Weight Percentage

| | | | | | | Vitamin | | |
|---|---|---|---|---|---|---|---|---|
| | | | | | | C | | |
| | Hydrolyzed | Glucosamine | Chondroitin | Hyaluronic | Methylsulfonylmethane | (ascorbic | | Omega |
| | Collagen | Sulfate | Sulfate | Acid | (MSM) | acid) | Manganese | 3 |
| Oral | 14.00% | 10.00% | 2.40% | 0.20% | 9.43% | 7.80% | 0.04% | 0.54% |
| Formulation | | | | | | | | |
| Embodiment | | | | | | | | |
| COSEQUIN ® | X | 42.35% | 7.06% | X | 29.41% | 1.79% | 0.29% | X |
| ASU PLUS | | | | | | | | |
| JOINT COMBO | X | X | 1.06% | X | 1.32% | 0.18% | 0.03% | 1.13% |
| STEADFAST ® | X | X | X | X | X | 2.00% | X | X |
| EQUINE | | | | | | | | |
| PLATINUM | X | 5.65% | X | 0.06% | 5.26% | X | 0.08% | 7.69% |
| PERFORMANCE ® | | | | | | | | |
| CJ | | | | | | | | |
| VITAFLEX ® PRO | X | X | X | X | 99.01% | X | X | X |
| MSM | | | | | | | | |
| JOINT AID | X | 11.76% | 2.35% | 0.12% | 8.82% | X | X | X |
| FLEX + MAX | X | X | 1.60% | 0.20% | 6.67% | X | X | X |
| ABSORBINE ® | | | | | | | | |
| JOINT ARMOR | X | 35.71% | 17.14% | 1.43% | X | X | 1.43% | X |

The following Table 3 presents a comparison of the various products with an alternative embodiment of the present oral formulation. In this alternative embodiment, the amount of hydrolyzed collagen may be reduced to about 3000 mg to about 5000 mg. in a 50 g oral dose preparation.

TABLE 3
Comparison of Alternative Oral Formulation Embodiment With Other Products

| | | | | | | Vitamin | | | |
|---|---|---|---|---|---|---|---|---|---|
| | Hydrolyzed | Glucosamine | Chondroitin | Hyaluronic | | C | | Omega | |
| | Collagen | Sulfate | Sulfate | Acid | Methylsulfonylmethane | (ascorbic | Manganese | 3 | Dose |
| | (mg) | (mg) | (mg) | (mg) | (MSM) (mg) | acid) (mg) | (mg) | (mg) | (g) |
| Oral | 3000 to | 5000 | 1200 | 100 | 4715 | 3900 | 20 | 270 | 50 |
| Formulation | 5000 | | | | | | | | |
| Embodiment | | | | | | | | | |
| COSEQUIN ® ASU | X | 14400 | 2400 | X | 10000 | 610 | 100 | X | 34 |
| PLUS | | | | | | | | | |
| JOINT COMBO | X | X | 600 | X | 750 | 104 | 16 | 638 | 56.7 |
| STEADFAST ® | X | X | X | X | X | 500 | X | X | 25 |
| EQUINE | | | | | | | | | |
| PLATINUM | X | 8820 | X | 90 | 8200 | X | 130 | 12000 | 156 |
| PERFORMANCE ® | | | | | | | | | |
| CJ | | | | | | | | | |
| VITAFLEX ® PRO | X | X | X | X | 10000 | X | X | X | 10.1 |
| MSM | | | | | | | | | |
| JOINT AID | X | 10000 | 2000 | 100 | 7500 | X | X | X | 85 |
| FLEX + MAX | X | X | 1200 | 150 | 5000 | X | X | X | 75 |
| ABSORBINE ® | | | | | | | | | |
| JOINT ARMOR | X | 5000 | 2400 | 200 | X | X | 200 | X | 14 |

### Example 8—Edible Biscuit and/or Food Bar Solid Formulation

The following illustrates an additional preparation of the formulations in a solid form, such as in a biscuit suitable for oral consumption, including consumption by both humans and veterinary animals.

In some embodiments, the solid form of the formulation may comprise a chewable supplement in a preparation comprising hydrolyzed collagen and other ingredients. The preparation may be suitable for animal consumption, including consumption by human, canine, equine, feline, or other animals.

In other embodiments, the solid form of the formulation may comprise an edible biscuit or other food product, such as a nutritional supplement bar. The biscuit or other food product will comprise a relatively high concentration of hydrolyzed collagen, along with the other ingredients as described herein. In this manner, it is envisioned that the biscuit or food bar will provide an amount of about 5,0000 mg to about 7,000 mg of hydrolyzed collagen to the human and/or veterinary animal upon consumption of the product.

The solid preparations may be prepared according to those routine and known formulation techniques known to those of skill in the art of consumable veterinary and/or human products.

### BIBLIOGRAPHY

The following references are incorporated herein in their entirety.
1. Goodman & Gilman's “The Pharmacological Basis of Therapeutics” eds. Hardman et al. Ninth Edition, McGraw-Hill Publishing, 1996
2. Camacho-Zambrano, M. M., et al. (2009). “A randomized controlled trial on the efficacy and safety of a food ingredient, collagen hydrolysate, for improving joint comfort”. International Journal of Food Sciences and Nutrition. 12: 1-15.
3. Osteoarthritis and Cartilage (2000) 8, 444-451, The effects of glucosamine derivatives on equine articular cartilage degradation in explant culture. J. I. Fenton, et al.
4. Forsyth R K, Brigden C V, Northrop A J. Double blind investigation of the effects of oral supplementation of combined glucosamine hydrochloride and chondroitin sulphate on stride characteristics of veteran horses. Equine Vet J (Suppl) 36:622-5, 2006.
5. Bartolucci, C., “Chondroprotective action of chondroitin sulfate,” Int. J. Tiss. Reac., XIII(6):311-317 (1991).
6. Manhart, D. & Scott, B. & Gibbs, P. & Coverdale, J. & Eller, E. & Honnas, C. & Hood, D. (2009). Markers of Inflammation in Arthritic Horses Fed Omega-3 Fatty Acids. The Professional Animal Scientist. 25. 10.15232/S1080-7446(15)30702-6.
7. V. Juliand, W. Martin-Rosset. (2005). The growing horse: nutrition and prevention of growth disorders. 190.
8. A. M. Johansson, et al. (2003) Hypomagnesemia in Hospitalized Horses. J. Vet. Intern. Med.; 17:860-867.
9. Butawan M, Benjamin R L, Bloomer R J. Methylsulfonylmethane: applications and safety of a novel dietary supplement. Nutrients. 2017; 9(pii):E290. doi: 10.3390/nu9030290.
10. Remington's Pharmaceutical Sciences, 16th Ed., Mac Publishing Company (1980).
11. Anon (1991) Lameness scale. Definition and classification of lameness. American Association of Equine Practitioners. 19.
12. Orsini J, Divers T. Management of special problems. Manual of Equine Emergencies, Treatment and Procedures. Philadelphia, Pa.: Saunders; 2003:749, 750.

What is claimed is:

1. A veterinary preparation comprising a combination of a glucosamine salt, a glycosaminoglycan, a sulfone, hydrolyzed collagen, a polysaccharide acid, an ascorbate salt, a transition metal, and a polyunsaturated fatty acid.

2. The veterinary preparation of claim 1 comprising an oral preparation.

3. The veterinary preparation of claim 1 comprising an oral preparation, said oral preparation comprising a powdered food additive.

4. The veterinary preparation of claim 1 comprising an intravenous preparation.

5. The preparation of claim 1 comprising an about 3000 milligram to an about 7000 milligram weight of hydrolyzed collagen per 50 gram weight of the preparation.

6. The preparation of claim 5 comprising about an about 3000 to an about 5000 milligram weight of hydrolyzed collagen per 50 gram weight of the preparation.

7. A method of enhancing collagen formation at a joint in a subject, the method comprising administering the preparation of claim 1 to the subject.

8. The method of claim 7, wherein the subject is a human or a veterinary animal.

9. The method of claim 8, wherein the veterinary animal is a canine.

10. The method of claim 8, wherein the veterinary animal is an equine.

11. The method of claim 7 wherein the preparation comprises an oral preparation.

12. The method of claim 7 wherein the subject is a human.

13. The method of claim 7 wherein the preparation is a powder, a liquid, or a solid preparation.

14. The method of claim 13 wherein the solid preparation is a biscuit or nutritional bar.
